(19) United States
(12) Patent Application Publication
(10) Pub. No.: US 2025/0303082 A1
(43) Pub. Date: Oct. 2, 2025

(54) RESPIRATORY SUPPORT APPARATUS

(71) Applicant: FUKUDA DENSHI CO., LTD., Tokyo (JP)

(72) Inventors: Mai KOBAYASHI, Tokyo (JP); Naoto SHIRAISHI, Tokyo (JP); Kai SAITO, Tokyo (JP)

(21) Appl. No.: 19/089,188

(22) Filed: Mar. 25, 2025

(30) Foreign Application Priority Data
Mar. 29, 2024 (JP) 2024-056593

(51) Int. Cl. A61M 16/00

(52) U.S. Cl. CPC A61M 16/0069; A61M 2205/3334; A61M 2205/3365

(57) ABSTRACT

A respiratory support apparatus of the present disclosure includes: a main body housing including intake and exhaust ports and forming a flow path of air, which includes a guidance path and a blower disposition chamber formed such that a flow-path cross-sectional area is expanded from a downstream end of the guidance path, from the intake port to the exhaust port; a blower disposed in the flow path and generating airflow to be delivered into a patient's airway; and a differential pressure sensor disposed to detect a state of the airflow, including a first measurement port disposed in the guidance path and a second measurement port disposed in the blower disposition chamber, which are disposed in the flow path, and measuring a differential pressure between the first measurement port and the second measurement port. The guidance path guides the air from the intake port to a position of the blower.

### CROSS REFERENCE TO RELATED APPLICATIONS

This application is entitled to and claims the benefit of Japanese Patent Application No. 2024-056593, filed on Mar. 29, 2024, the disclosure of which including the specification, drawings and abstract is incorporated herein by reference in its entirety.

### TECHNICAL FIELD

The present disclosure relates to a respiratory support apparatus.

### BACKGROUND ART

Conventionally, a respiratory support apparatus that supplies airflow to a patient's airway, such as a continuous positive airway pressure (CPAP) apparatus, has been known. Note that, the CPAP apparatus is an apparatus used for CPAP therapy (also referred to as a sleep apnea treatment apparatus). CPAP therapy is a treatment method that prevents apnea in a patient during sleep, who has symptoms of obstructive sleep apnea, by continuously supplying air to the airway of the patient to widen the airway.

A respiratory support apparatus of this type generally includes: a blower that generates airflow; and a built-in board for controlling the blower, in a main body housing, and is configured to be capable of adjusting the airflow with the blower such that the flow rate becomes suitable for widening a patient's airway. Further, as a respiratory support apparatus of this type, a respiratory support apparatus has also been developed that includes a humidifier for adjusting the temperature and humidity of the airflow to be supplied to a patient.

Such a respiratory support apparatus is described in, for example, Japanese Patent Application Laid-Open No. 2023-071739.

Incidentally, in a respiratory support apparatus of this type, a flow sensor is conventionally disposed in a flow path of air in a housing of an apparatus main body to measure the respiratory flow of a patient and control the operation (that is, the rotation speed) of the blower based on the respiratory flow. As the flow sensor, a differential pressure sensor is generally used, and the flow rate of airflow is measured based on a pressure difference between two points in the flow path.

Note that, a respiratory flow refers to airflow generated by a patient's breathing (the same applies hereinafter). The magnitude of the flow rate of airflow in a respiratory flow is observed as a respiratory waveform. It is generally known that a respiratory waveform includes, in addition to a low-frequency component accompanying a normal respiratory motion, a high-frequency component of approximately 10 to 100 Hz due to snoring.

In the respiratory support apparatus according to the related art, there is room for improvement in the measurement accuracy of a patient's respiratory waveform. This means that, in the respiratory support apparatus according to the related art, there is a possibility that the control of the operation (that is, the rotation speed) of the blower in accordance with a patient's breathing cannot be appropriately performed.

Accordingly, an object of the present invention is to provide a respiratory support apparatus that makes it possible to capture a patient's respiratory flow more accurately.

### SUMMARY

A main aspect of the present invention for solving the above-described challenges is a respiratory support apparatus that includes:
a main body housing that includes an intake port and an exhaust port and forms a flow path of air from the intake port to the exhaust port;
a blower that is disposed in the flow path and generates airflow to be delivered into an airway of a patient; and
a differential pressure sensor that is disposed to detect a state of the airflow, includes a first measurement port and a second measurement port which are disposed in the flow path, and measures a differential pressure between the first measurement port and the second measurement port.

The flow path includes:
a guidance path that guides the air from the intake port to a position of the blower; and
a blower disposition chamber that is formed such that a flow-path cross-sectional area is expanded from a downstream end of the guidance path.

The first measurement port is disposed in the guidance path, and
the second measurement port is disposed in the blower disposition chamber.

### BRIEF DESCRIPTION OF THE DRAWINGS

FIG. 1 is a diagram illustrating how a CPAP apparatus is attached to a patient;

FIG. 2A is a perspective view of the CPAP apparatus according to an embodiment of the present invention as seen from obliquely above;

FIG. 2B is a perspective view of the CPAP apparatus according to the embodiment of the present invention as viewed from obliquely above;

FIG. 3 is an exploded perspective view of the CPAP apparatus according to the embodiment of the present invention;

FIG. 4 is a schematic diagram illustrating a route of airflow;

FIG. 5 is a block diagram illustrating the configuration of the CPAP apparatus according to the embodiment of the present invention;

FIG. 6A is a diagram illustrating a configuration of a flow path and a disposition configuration of a differential pressure sensor in the CPAP apparatus according to the embodiment of the present invention;

FIG. 6B is a diagram illustrating the configuration of the flow path and the disposition configuration of the differential pressure sensor in the CPAP apparatus according to the embodiment of the present invention;

FIG. 7A is a diagram illustrating a configuration of a flow path and a disposition configuration of a differential pressure sensor in a CPAP apparatus according to a comparative example;

FIG. 7B is a diagram illustrating a configuration of a flow path and a disposition configuration of a differential pressure sensor in the CPAP apparatus according to the comparative example;

FIG. 8 is a schematic diagram illustrating a mechanism for detecting a high- frequency component of a flow rate change in air by the differential pressure sensor according to the embodiment of the present invention;

FIG. 9 is a schematic diagram illustrating an aspect of flow rate measurement according to the related art; and

FIG. 10 is a diagram describing a challenge in the flow rate measurement according to the related art.

### DESCRIPTION OF EMBODIMENTS

Hereinafter, a preferred embodiment of the present invention will be described in detail with reference to the accompanying drawings. Note that, in the present specification and drawings, components having substantially the same functions are denoted by the same reference signs, and redundant descriptions are omitted thereby.

Hereinafter, a CPAP apparatus (hereinafter, referred to as “CPAP apparatus 100”) will be described as a preferred application example of the respiratory support apparatus according to the present invention. Note that, the respiratory support apparatus according to the present invention can be applied not only to a CPAP apparatus but also to an adaptive servo ventilation (ASV) apparatus or a Nasal High Flow (NHF) apparatus.

### <1> Configuration of CPAP Apparatus 100 in Embodiment

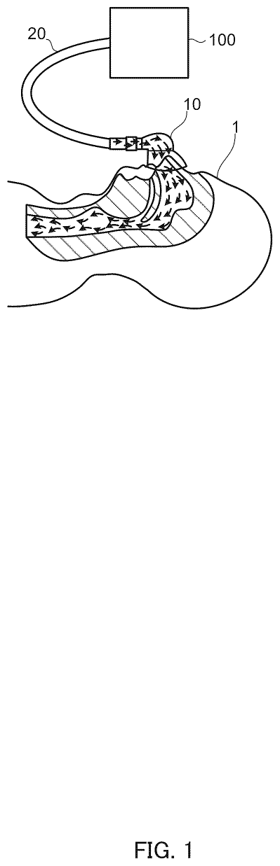

FIG. 1 is a diagram illustrating how CPAP apparatus 100 is attached to patient 1. As illustrated in FIG. 1, CPAP apparatus 100 includes mask 10 and tube 20, and an apparatus main body of CPAP apparatus 100 is connected to mask 10, which is put on the face of patient 1 with sleep apnea syndrome, via tube 20, and delivers positive pressure airflow for expanding the upper airway of patient 1 to the upper airway.

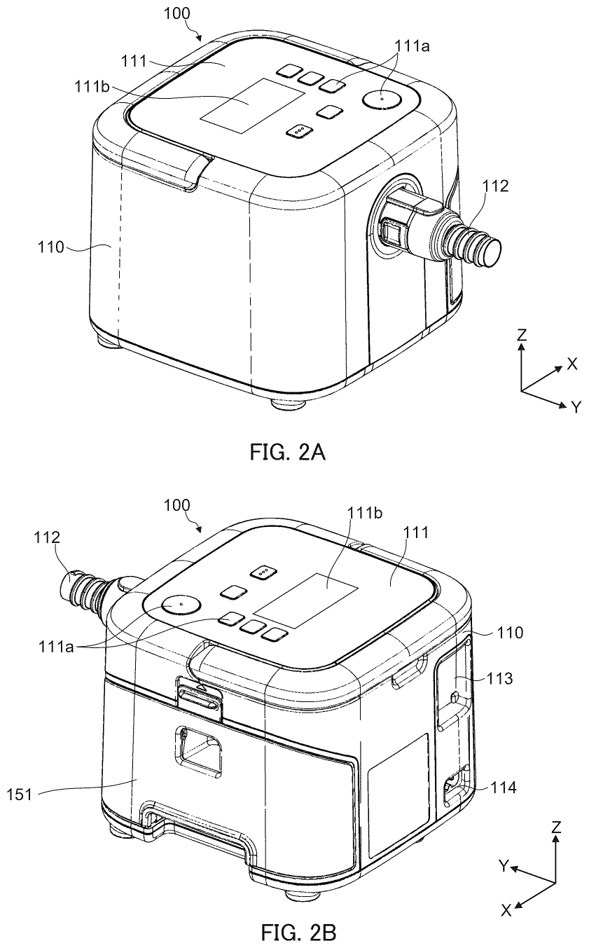

FIGS. 2A and 2B are perspective views of CPAP apparatus 100 as seen from obliquely above. Here, in FIGS. 2A and 2B, the +Z direction indicates the upward direction of CPAP apparatus 100, and the −Z direction indicates the downward direction of CPAP apparatus 100. Further, the +Y direction indicates the frontward direction of CPAP apparatus 100, and the −Y direction indicates the rearward direction of CPAP apparatus 100. Further, the +X direction indicates the left direction of CPAP apparatus 100, and the −X direction indicates the right direction of CPAP apparatus 100.

As can be seen in FIG. 2A, tube connector 112 to which tube 20 (see FIG. 1) is connected protrudes from the front side surface of accommodation case 110 of CPAP apparatus 100. Further, operation panel 111 is provided in an upper portion of accommodation case 110. Operation panel 111 is provided with operation inputter 111a including an operation button or the like and display 111b.

As can be seen in FIG. 2B, intake port 113 and power connector 114 are provided on the rear side surface of accommodation case 110. An AC power supply is inputted into power connector 114 via a power cable. Further, water tank 151 is detachably attached to the right side surface of accommodation case 110.

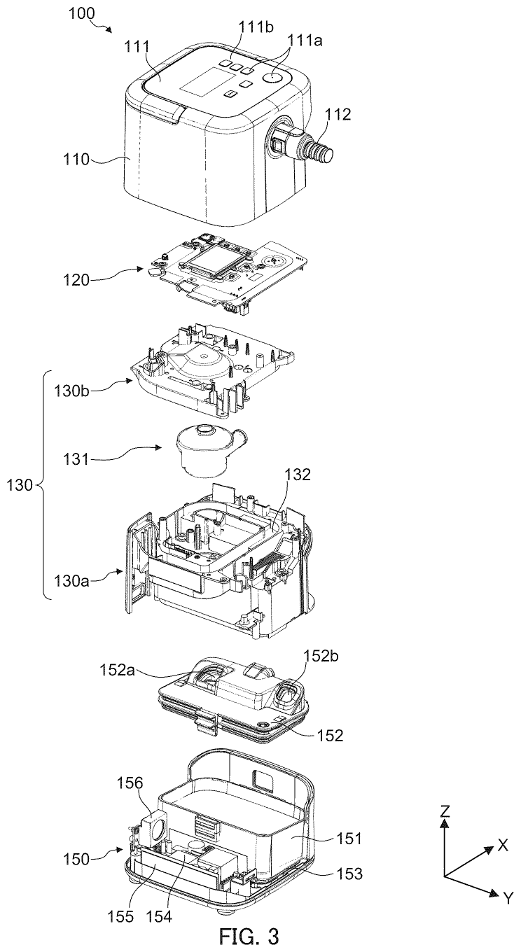

FIG. 3 is an exploded perspective view of CPAP apparatus 100.

CPAP apparatus 100 is configured to include accommodation case 110, circuit board 120, flow path case 130, and base 150.

Note that, in CPAP apparatus 100 according to the present embodiment, a main body housing of CPAP apparatus 100 is constituted by accommodation case 110, flow path case 130, and base 150 (hereinafter, the main body housing will also be referred to as “main body housing 100A”).

Accommodation case 110 has a rectangular tube shape, and accommodates circuit board 120, flow path case 130, and the like by being coupled to base 150 from above. Further, operation panel 111 is disposed in the upper portion of accommodation case 110.

As described above, accommodation case 110 includes tube connector 112, intake port 113, and power connector 114. Note that, tube connector 112 constitutes an exhaust port of main body housing 100A.

Circuit board 120 is provided with a microcomputer, a memory, various driver circuits, and the like.

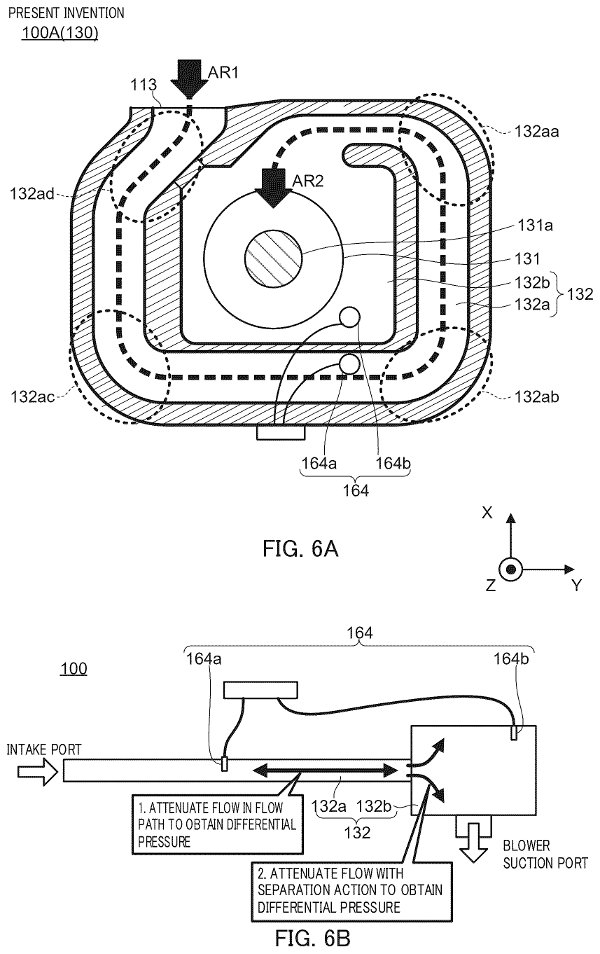

Flow path case 130 is configured by fitting lower case 130a and upper case 130b together. Blower 131 is disposed inside flow path case 130. In flow path case 130, flow path 132 (to be described later with reference to FIG. 6) is formed through which air sucked in through intake port 113 passes, and blower 131 is disposed in flow path 132. Note that, blower 131 gives energy to the air sucked in through intake port 113 and flowing through flow path 132, increases the pressure, increases the velocity, and sends the air to a side of a humidifier constituted by water tank 151 or the like.

In base 150, water tank 151 is disposed which is detachable. In lid 152 of water tank 151, air inlet port 152a and air exhaust port 152b are formed. Air inlet port 152a communicates with flow path 132 in flow path case 130. Air exhaust port 152b communicates with tube connector 112.

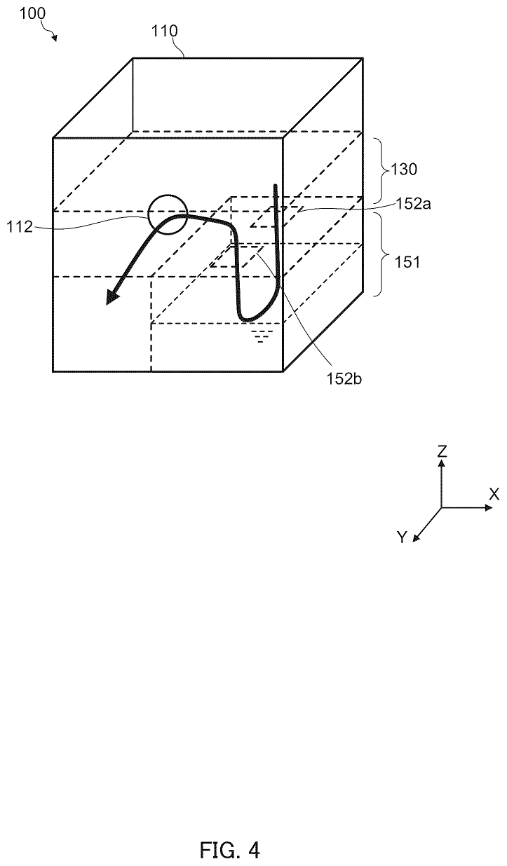

Thereby, as can be understood from the schematic diagram in FIG. 4, the airflow (the arrow in FIG. 4) generated by blower 131 passes through flow path 132 in flow path case 130, then enters water tank 151 through air inlet port 152a, is discharged from water tank 151 through air exhaust port 152b, and is supplied to patient 1 via tube connector 112.

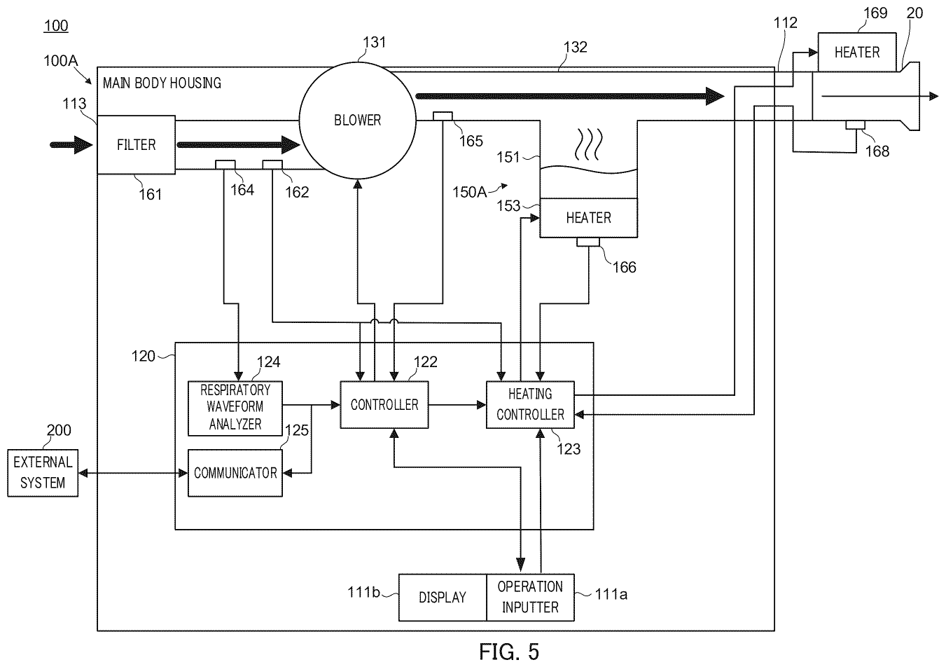

Heater 153 is provided on a side of the lower surface of water tank 151. The water in water tank 151 is heated by heater 153, resulting in a high humidity state in water tank 151. Thus, the airflow to be supplied to the patient is humidified in water tank 151. The drying of the airway of patient 1 due to the airflow is suppressed thereby. That is, in CPAP apparatus 100 according to the present embodiment, a humidifier that humidifies the airflow to be supplied to patient 1 is constituted by water tank 151, lid 152, and heater 153 (hereinafter, the humidifier will also be referred to as “humidifier 150A”) (see FIG. 5).

Further, AC/DC converter 154 is provided in base 150. AC/DC converter 154 receives an input of an external AC power supply from a power cord (not illustrated) connected to power connector 114 (FIG. 2B), converts the AC power supply into a DC power supply, and supplies the converted DC power supply to circuit board 120 and the like.

The lower side and both the left and right sides of a plurality of circuit components constituting AC/DC converter 154 are covered by sheet metal member 155 which has a U-shaped cross section cut in the XZ plane. Sheet metal member 155 extends in the Y direction. Fan 156 for cooling AC/DC converter 154 is provided on a side of one end of sheet metal member 155. Fan 156 is provided at a position facing AC/DC converter 154.

Thereby, AC/DC converter 154 is efficiently cooled by the wind of fan 156. Further, electromagnetic noise generated by AC/DC converter 154 is shielded by sheet metal member 155, thereby reducing the influence of the electromagnetic noise on other circuit boards and the like.

As described above, in CPAP apparatus 100 according to the present embodiment, the air sucked in through intake port 113 passes through flow path 132 and blower 131 in flow path case 130, and humidifier 150A, and is supplied to patient 1 via tube connector 112.

FIG. 5 is a block diagram illustrating the configuration of CPAP apparatus 100.

In flow path 132 of CPAP apparatus 100, filter 161, temperature/humidity sensor 162, differential pressure sensor 164, and pressure sensor 165 are provided in addition to blower 131. Further, temperature sensor 166 is attached to heater 153 that heats water tank 151.

Circuit board 120 is provided with controller 122, heating controller 123, respiratory waveform analyzer 124, communicator 125, and the like. In other words, circuit components for implementing each function of controller 122, heating controller 123, respiratory waveform analyzer 124, and communicator 125 are mounted in circuit board 120.

Note that, controller 122, heating controller 123, and respiratory waveform analyzer 124 are each constituted by, for example, a microcomputer including a central processing unit (CPU), a read only memory (ROM), and a random access memory (RAM). The CPU reads a program corresponding to a processing content from the ROM, develops the program in the RAM, and cooperates with the developed program to implement each function of controller 122, heating controller 123, and respiratory waveform analyzer 124. Note that, controller 122, heating controller 123, and respiratory waveform analyzer 124 may be formed entirely or partially of a hard-wired circuit such as an application specific integrated circuit (ASIC) or a field-programmable gate array (FPGA).

When blower 131 operates, external air enters flow path 132 via intake port 113 and filter 161. Then, the temperature and humidity of the air in flow path 132 are measured by temperature/humidity sensor 162, and the measured temperature and humidity are sent to controller 122 and heating controller 123. Further, a heating set value and a humidification set value (for example, a target temperature and a target humidity) from operation inputter 111a are inputted into heating controller 123, and temperature information of heater 153 from temperature sensor 166 is inputted into heating controller 123.

Heating controller 123 controls heater 153 based on information on the temperature and humidity measured by temperature/humidity sensor 162, the heating set value and the humidification set value from operation inputter 111a set by the user, and the temperature information of heater 153 from temperature sensor 166. For example, heating controller 123 controls heater 153 such that the temperature and humidity of the airflow to be supplied to patient 1 approach the heating set value and the humidification set value, respectively.

Further, temperature information from temperature sensor 168 provided in tube 20 is inputted into heating controller 123. Heating controller 123 controls heater 169 provided in tube 20 based on this temperature information to suppress condensation in tube 20.

Differential pressure sensor 164 is disposed as a flow sensor that measures the flow rate of airflow flowing through flow path 132. Differential pressure sensor 164 measures a pressure difference in airflow between two points of first measurement port 164a and second measurement port 164b (to be described later with reference to FIGS. 6A and 6B), and sends the measurement result to respiratory waveform analyzer 124. Note that, differential pressure sensor 164 may be a differential pressure sensor that performs differential pressure measurement based on a thermal flow measurement principle, a differential pressure sensor that performs differential pressure measurement using a pressure-sensitive element, or a differential pressure sensor that performs differential pressure measurement in another form.

Note that, differential pressure sensor 164 is disposed at an appropriate position on the upstream side of blower 131. This is because air turbulence occurs even when differential pressure sensor 164 is too close to blower 131 or even when differential pressure sensor 164 is away from blower 131 and is in the vicinity of intake port 113 (details will be described later with reference to FIGS. 6A and 6B). Further, on the downstream side of blower 131, the airflow is pressurized by blower 131 and it is not possible to measure the differential pressure with high accuracy by differential pressure sensor 164, and thus, differential pressure sensor 164 is disposed on the upstream side of blower 131.

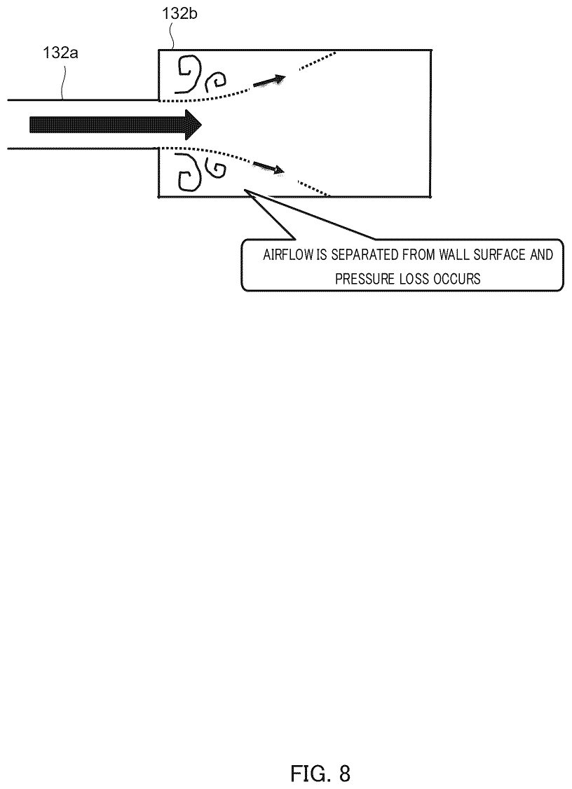

Respiratory waveform analyzer 124 detects the state of airflow based on the measurement data of differential pressure sensor 164. Here, “detecting the state of airflow” means detecting the flow rate of airflow or an airflow vibration. That is, since an airflow vibration is superimposed on a flow rate waveform, respiratory waveform analyzer 124 can also detect a density change due to a snoring component (high-frequency component) of breathing (to be described later with reference to FIG. 8).

Specifically, for example, respiratory waveform analyzer 124 first obtains flow rate information on airflow from a differential pressure as measurement data obtained from differential pressure sensor 164 by using a control map or the like. Then, respiratory waveform analyzer 124 detects the respiratory flow (that is, the respiratory waveform) of patient 1 based on a temporal change in the flow rate of the airflow. Then, respiratory waveform analyzer 124 performs, for example, frequency analysis (for example, FFT analysis) or the like on the detected respiratory flow (respiratory waveform), and sends the analysis result (for example, signal intensity for each frequency) as respiration information to controller 122. Further, respiratory waveform analyzer 124 sends the respiration information to communicator 125, for example.

Note that, for example, respiratory waveform analyzer 124 may capture a low-frequency component (normal respiratory motion component) and a high-frequency component (snoring component) by performing frequency analysis on the respiratory waveform, and may control the rotation of blower 131 based on the waveform change for each of the frequency components.

Pressure information on the pressure in flow path 132 measured by pressure sensor 165 is sent to controller 122. Further, pressure setting information (for example, target pressure) from operation inputter 111a is inputted into controller 122.

Controller 122 controls the flow rate of the airflow to be supplied to patient 1 by controlling the rotation of blower 131 based on the pressure information measured by pressure sensor 165, the respiration information from respiratory waveform analyzer 124, and the pressure setting information from operation inputter 111a set by the user.

For example, in a case where a high-frequency component with a large intensity (that is, snoring) is detected, controller 122 considers that there is airway resistance (that is, the airway is narrowed and is about to be blocked) and increases the pressure (that is, increases the rotation speed of blower 131) to widen the airway. Further, for example, in a case where a low-frequency flow rate change due to the respiratory motion becomes small, controller 122 increases the pressure (that is, increases the rotation speed of blower 131) to widen the airway, assuming that the patient is experiencing apnea or hypopnea.

Further, controller 122 specifies the usage temperature of blower 131 based

on the information on the temperature measured by, for r example, temperature/humidity sensor 162, and limits or stops the operation of humidifier 150A to prevent deterioration of blower 131 in a case where the usage temperature exceeds a threshold temperature. Note that, for example, 50 degrees Celsius is set as the threshold temperature.

Communicator 125 performs communication with external system 200. For example, the respiration information obtained by respiratory waveform analyzer 124 is transmitted to external system 200 via communicator 125. Thereby, a medical professional who is at a location away from CPAP apparatus 100 can know the breathing state of patient 1, and can know, for example, that patient 1 is experiencing apnea.

### <2> Disposition Configuration of Differential Pressure Sensor 164

Next, details of a configuration of flow path 132 in main body housing 100A and a disposition configuration of differential pressure sensor 164 according to the present embodiment will be described.

First, a challenge for the CPAP apparatus according to the related art will be described.

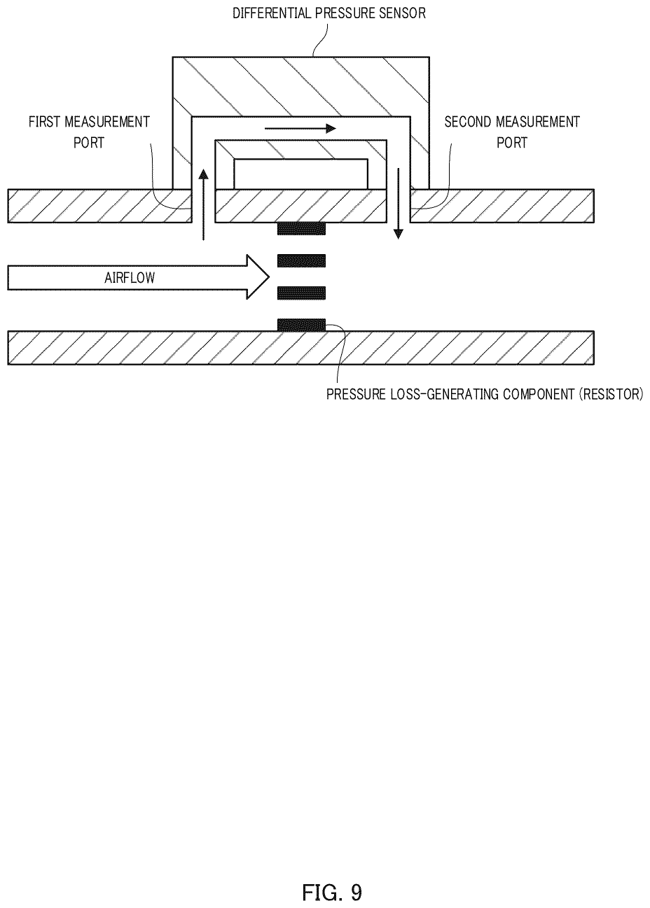

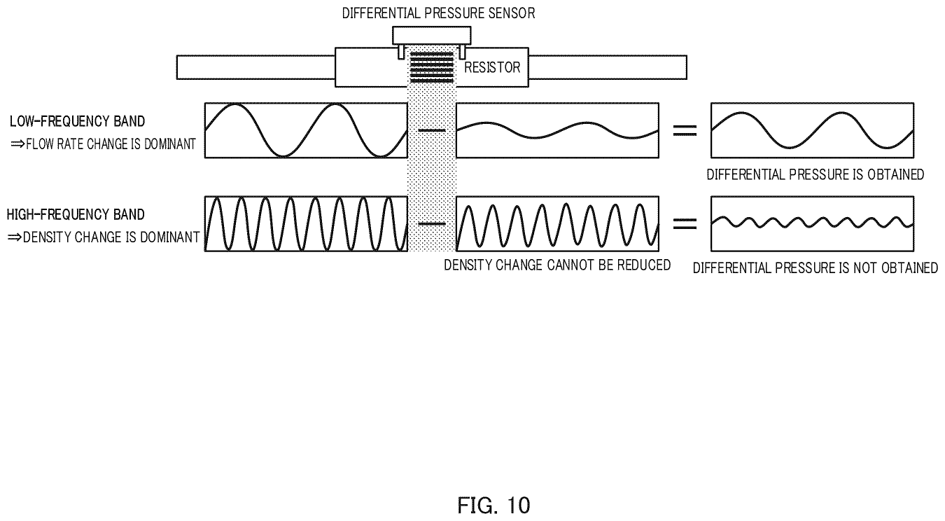

FIG. 9 is a schematic diagram of an aspect of flow rate measurement according to the related art. FIG. 10 is a diagram describing a challenge in the flow rate measurement according to the related art.

Conventionally, in flow rate measurement using a differential pressure sensor, a technique has been adopted in which two measurement ports of the differential pressure sensor are disposed in a flow path, a resistor having a plurality of walls is installed between the two measurement ports, the flow of gas is attenuated by frictional resistance on side walls thereof (that is, a pressure loss is generated), and the flow rate is determined based on the differential pressure between before and after the attenuation.

However, as a result of intensive studies by the present inventors, it has been found that the conventional technique using such a resistor cannot detect a signal of a high-frequency component of a respiratory flow (for example, a high-frequency component of approximately 10 to 100 Hz due to snoring) with high accuracy.

As a property of a gas, it is generally known that when the flow velocity increases, the gas is transmitted through a flow path as a density change. That is, the above-described phenomenon is presumed to occur due to the fact that a flow rate change in airflow (meaning a temporal change in airflow) is expressed as the flow rate change itself for a low-frequency component, but is expressed as a density change for a high-frequency component.

That is, the resistor used in the related art is considered to be capable of reducing a flow velocity change (that is, a flow rate change) in airflow due to frictional resistance, but is considered to allow a density change in airflow to pass therethrough (see FIG. 10). For this reason, in the related art in which the flow rate of airflow is measured using a resistor, it is not possible to sufficiently obtain a differential pressure of airflow due to a high-frequency component of a respiratory flow between two measurement ports of a differential pressure sensor. As a result, in the related art, it is not possible to sufficiently detect a signal of a high-frequency component of approximately 10 to 100 Hz due to snoring or the like.

This means that the respiratory support apparatus according to the related art cannot sufficiently grasp a breathing disorder, airway resistance or the like, such as snoring, and therefore may not be capable of appropriately controlling the operation (that is, the rotation speed) of the blower in accordance with the breathing of the patient.

CPAP apparatus 100 according to the present embodiment employs a disposition configuration of differential pressure sensor 164 in consideration of the above-described challenge.

FIGS. 6A and 6B are diagrams illustrating a disposition configuration of differential pressure sensor 164 in CPAP apparatus 100 according to the present embodiment. FIG. 6A illustrates flow path 132 formed in flow path case 130 in a plan view. Further, FIG. 6B schematically illustrates the disposition positions of first and second measurement ports 164a and 164b of differential pressure sensor 164 in flow path 132.

FIG. 8 is a schematic diagram illustrating a mechanism for detecting a high-frequency component of a flow rate change in air by differential pressure sensor 164 according to the present embodiment.

Flow path 132 includes: guidance path 132a that guides air introduced into intake port 113; and blower disposition chamber 132b that is formed to be connected to the downstream end of guidance path 132a.

Guidance path 132a includes at least one of bends 132aa to 132ad to guide air from intake port 113 to the position of blower 131 (that is, blower disposition chamber 132b) such that the air largely detours in main body housing 100A. Guidance path 132a according to the present embodiment includes four bends 132aa to 132ad such that guidance path 132a passes through positions at four corners in main body housing 100A in a plan view. That is, guidance path 132a according to the present embodiment guides air such that the air circulates in main body housing 100A along the side walls in main body housing 100A, instead of guiding the air in a straight-line manner with the shortest distance from intake port 113 to the position of blower 131 in main body housing 100A. The reason why this configuration is adopted in CPAP apparatus 100 according to the present embodiment is to increase the length of flow path 132 from intake port 113 to the position of blower 131 and to reduce the degree to which noise generated at blower 131 leaks to the outside. Further, it is also possible to reduce noise by disposing a sound-absorbing material on the route thereof and increasing the length thereof.

Blower disposition chamber 132b is a region in which blower 131 is disposed, and has, for example, a substantially rectangular shape in an XY plane view. Blower disposition chamber 132b is formed, in an XY plane view, by huge expansion of a flow-path cross-sectional area (which means a cross-sectional area in the direction orthogonal to the flow direction of airflow in flow path 132; the same applies hereinafter) from the downstream end of guidance path 132a. More specifically, blower disposition chamber 132b is formed by huge expansion of the flow path width in the +Y direction from the downstream end of guidance path 132a.

Blower 131 is disposed in blower disposition chamber 132b such that suction port 131a (the hatched region in FIG. 6A) of blower 131 is located in the center of blower disposition chamber 132b in a plan view. Note that, CPAP apparatus 100 according to the present embodiment has a configuration in which blower 131 includes suction port 131a that opens in a direction (that is, the Z-axis direction) intersecting with the air guidance direction of guidance path 132a, from the viewpoint of securing a long flow path length of guidance path 132a. Blower disposition chamber 132b is formed to bend air coming from guidance path 132a in the direction intersecting with the guidance direction of guidance path 132a, and is formed to have a widened flow-path cross-sectional area.

As described above, the air (black arrow AR1) introduced through intake port 113 is sucked into suction port 131a of blower 131 (black arrow AR2) via guidance path 132a and blower disposition chamber 132b, and is sent to water tank 151 (humidifier 150A) via air inlet port 152a through an exhaust port (not illustrated) of blower 131. At this time, when air is sucked into suction port 131a of blower 131, the air is sucked in from the +Z direction toward the −Z direction.

In such a flow path configuration in CPAP apparatus 100 according to the present embodiment, first measurement port 164a of differential pressure sensor 164 is disposed in guidance path 132a, and second measurement port 164b of differential pressure sensor 164 is disposed in blower disposition chamber 132b. In addition, differential pressure sensor 164 is configured to measure a pressure difference in airflow between two points which are the position of first measurement port 164a and the position of second measurement port 164b.

The reason why this configuration is adopted in CPAP apparatus 100 according to the present embodiment is to enable differential pressure sensor 164 to detect, in addition to a low-frequency component of a respiratory flow, a high-frequency component of the respiratory flow. The principle of detecting a high-frequency component of a respiratory flow in CPAP apparatus 100 according to the present embodiment is as illustrated in FIG. 8.

That is, in CPAP apparatus 100 according to the present embodiment, a change in the flow-path cross-sectional area from guidance path 132a to blower disposition chamber 132b has an aspect of huge expansion in a step-like manner. Thereby, when airflow enters blower disposition chamber 132b from guidance path 132a, the airflow is separated from the wall surface. In addition, a pressure loss occurs thereby. The pressure loss generated by the separation of the airflow causes not only a flow velocity change in the airflow but also a density change in the airflow. For this reason, differential pressure sensor 164 is also capable of detecting a high-frequency component of a flow rate change in airflow by utilizing such a pressure loss.

Note that, when a high-frequency component of a flow rate change in airflow is detected by differential pressure sensor 164, it is preferable to separate the airflow from the wall surface, and in order to cause the airflow to separate from the wall surface, the aspect of the change in the flow-path cross-sectional area from guidance path 132a to blower disposition chamber 132b may not be in a step-like manner.

Note that, first measurement port 164a of differential pressure sensor 164 is preferably disposed at a position in guidance path 132a, where the position is a predetermined distance away from a connection position between guidance path 132a and blower disposition chamber 132b toward a side of intake port 113. Such a predetermined distance is, for example, 10 cm to 40 cm. That is, the disposition position of first measurement port 164a is preferably on the upstream side of the connection position between guidance path 132a and blower disposition chamber 132b, and on the upstream side of at least bend 132aa on the most downstream side. However, in the vicinity of intake port 113, airflow disturbance often occurs, and thus, the disposition position of first measurement port 164a is preferably on a side of blower 131 (on the downstream side) with respect to an intermediate position between intake port 113 and blower 131 in guidance path 132a. Further, the disposition position of first measurement port 164a is preferably a position in a straight-line portion in guidance path 132a, rather than the positions of bends 132aa to 132ad in guidance path 132a. In other words, first measurement port 164a and second measurement port 164b are disposed so as to hold bend 132aa therebetween on guidance path 132a.

Thereby, airflow causes a pressure loss due to frictional resistance (particularly, frictional resistance when passing through bend 132aa) when passing through guidance path 132a between first measurement port 164a and second measurement port 164b. That is, it is also possible to improve the measurement accuracy of a low-frequency component (that is, a low-frequency component of less than 10 Hz due to breathing) of a flow rate change in airflow in differential pressure sensor 164 thereby.

Further, second measurement port 164b of differential pressure sensor 164 is more preferably disposed, in blower disposition chamber 132b, on the side opposite to the connection position between guidance path 132a and blower disposition chamber 132b (that is, in blower disposition chamber 132b, in the vicinity of the surface facing the connection position between guidance path 132a and blower disposition chamber 132b). Thereby, it is possible to suppress mixing of noise into a measurement result due to disturbance of airflow generated at a position where the airflow flows from guidance path 132a into blower disposition chamber 132b.

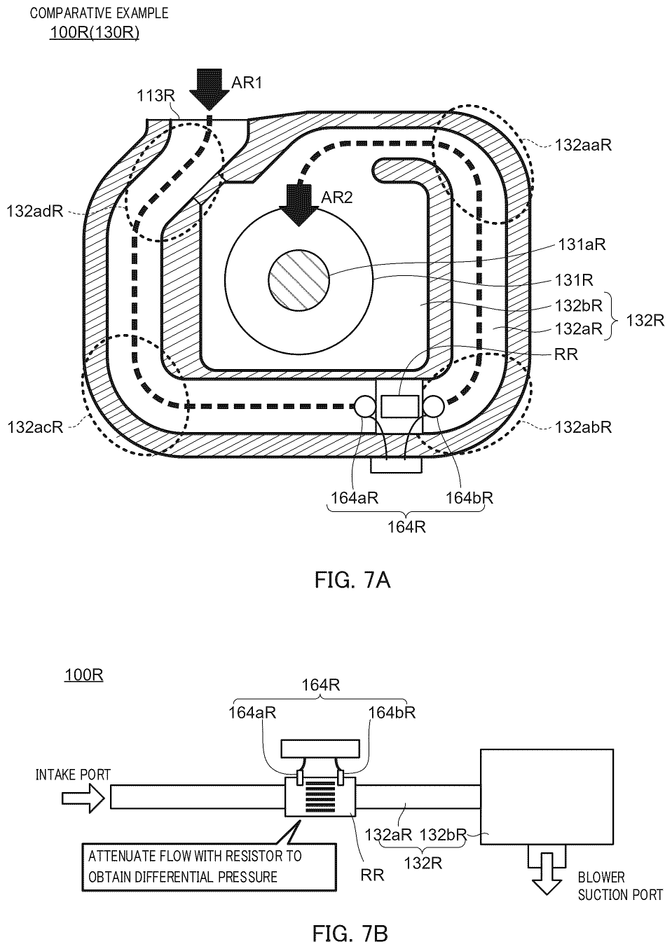

FIGS. 7A and 7B are diagrams illustrating a disposition configuration of differential pressure sensor 164R in CPAP apparatus 100R according to a comparative example. FIG. 7A illustrates flow path 132R formed in flow path case 130R in a plan view. Further, FIG. 7B schematically illustrates the disposition positions of first and second measurement ports 164aR and 164bR of differential pressure sensor 164R in flow path 132R. Note that, CPAP apparatus 100R according to the comparative example has an apparatus configuration of a CPAP apparatus, which is assumed when the flow rate measurement technique according to the related art is applied to CPAP apparatus 100 according to the present embodiment.

Given the difference between CPAP apparatus 100 according to the present embodiment and CPAP apparatus 100R according to the comparative example (see FIGS. 7A and 7B), CPAP apparatus 100 according to the present embodiment is configured not to include resistor RR in guidance path 132a.

This is because CPAP apparatus 100 according to the present embodiment employs a technique that secures the differential pressure between first measurement port 164a and second measurement port 164b by utilizing a pressure loss, which is generated by the separation of airflow from the wall surface when the airflow enters blower disposition chamber 132b from guidance path 132a, and a pressure loss due to frictional resistance when airflow passes through guidance path 132a. Further, since such a technique does not use resistor RR, there is an additional effect that a decrease in the output of blower 131 due to the airflow obstruction of resistor RR can be suppressed.

### <3> Summary

As described above, respiratory support apparatus 100 according to the present embodiment includes: main body housing 100A that includes intake port 113 and an exhaust port and forms flow path 132 of air from intake port 113 to the exhaust port; blower 131 that is disposed in flow path 132 and generates airflow to be delivered into an airway of a patient; and differential pressure sensor 164 that measures a flow rate of the air flowing through flow path 132, in which flow path 132 includes: guidance path 132a that guides the air from intake port 113 to a position of blower 131; and blower disposition chamber 132b that is formed such that a flow-path cross-sectional area is expanded from a downstream end of guidance path 132a, and differential pressure sensor 164 includes first measurement port 164a disposed in guidance path 132a and second measurement port 164b disposed in blower disposition chamber 132b, and measures the flow rate of the airflow based on a pressure difference in airflow between two points of a position of first measurement port 164a and a position of second measurement port 164b.

Accordingly, CPAP apparatus 100 according to the present embodiment makes it possible to capture the respiratory waveform of a patient more accurately.

Thereby, it is possible to appropriately perform control of the operation (that is, the rotation speed) of blower 131 in accordance with the respiratory flow of a patient. Note that, CPAP apparatus 100 may, for example, capture a low-frequency component (normal respiratory motion component) and a high-frequency component (snoring component) from a respiratory waveform, respectively, and control the rotation of blower 131 based on a waveform change for each of the frequency components.

The present invention is not limited to the above embodiment, and is applicable to various modification aspects.

For example, in the above embodiment, an aspect in which accommodation case 110, flow path case 130, and base 150 constitute main body housing 100A of CPAP apparatus 100 has been indicated. However, these do not necessarily need to be separable in terms of realizing CPAP apparatus 100 according to the present invention.

Further, in the above embodiment, an aspect in which blower 131 increases the pressure of air flowing through flow path 132 has been indicated. However, in terms of realizing CPAP apparatus 100 according to the present invention, blower 131 does not necessarily need to discharge high-pressure airflow, and may discharge low-pressure airflow, such as that of a so-called fan.

Any of the embodiment described above is only illustration of an exemplary embodiment for implementing the present invention, and the technical scope of the present invention should not be construed limitedly thereby. That is, the present invention can be implemented in various forms without departing from the spirit or main features thereof.

At least following matters will be apparent from the description of the present specification and the accompanying drawings.

The present specification discloses a respiratory support apparatus including: a main body housing that includes an intake port and an exhaust port and forms a flow path of air from the intake port to the exhaust port; a blower that is disposed in the flow path and generates airflow to be delivered into an airway of a patient; and a differential pressure sensor that is disposed to detect a state of the airflow, includes a first measurement port and a second measurement port which are disposed in the flow path, and measures a differential pressure between the first measurement port and the second measurement port. The flow path includes: a guidance path that guides the air from the intake port to a position of the blower; and a blower disposition chamber that is formed such that a flow-path cross-sectional area is expanded from a downstream end of the guidance path. The first measurement port is disposed in the guidance path, and the second measurement port is disposed in the blower disposition chamber.

It is possible to capture the respiratory waveform of a patient more accurately thereby. Further, it is possible to appropriately perform control of the operation (that is, the rotation speed) of the blower in accordance with the respiratory flow of a patient thereby.

In the respiratory support apparatus, the first measurement port is preferably disposed at a position in the guidance path, where the position is a predetermined distance away from a connection position between the guidance path and the blower disposition chamber toward a side of the intake port.

Thereby, it is also possible to improve the measurement accuracy of a low-frequency component (that is, a low-frequency component of less than 10 Hz due to breathing) of a flow rate change in airflow in the differential pressure sensor. That is, it is possible to capture the respiratory waveform of a patient more accurately thereby.

Further, in the respiratory support apparatus, the second measurement port is preferably disposed at a position in the blower disposition chamber, where the position is a position on a side opposite to the connection position between the guidance path and the blower disposition chamber.

Thereby, it is possible to suppress mixing of noise into a measurement result due to disturbance of airflow generated at a position where the airflow flows from the guidance path into the blower disposition chamber. That is, it is possible to capture the respiratory waveform of a patient more accurately thereby.

Further, the respiratory support apparatus preferably further includes a controller that controls a rotation speed of the blower based on a respiratory waveform of the patient, where the respiratory waveform is detected from a measurement result of the differential pressure sensor.

Thereby, it is possible to appropriately perform control of the operation (that is, the rotation speed) of the blower in accordance with the respiratory flow of a patient.

Further, in the respiratory support apparatus, the guidance path preferably guides the air from the intake port to the position of the blower such that the air largely detours in the main body housing.

Thereby, it is possible to reduce the degree to which noise generated at the blower leaks to the outside.

Further, in the respiratory support apparatus, the blower preferably includes a suction port that opens in a direction intersecting with a guidance direction of the air in the guidance path, and the blower disposition chamber is preferably formed so as to bend the air, which comes from the guidance path, in the direction intersecting with the guidance direction.

Thereby, it is possible to suppress an increase in the size of the apparatus while securing a long flow path length. That is, it is possible to deliver airflow in accordance with the respiratory flow of a patient to the patient thereby. Further, it is possible to perform a decrease in the size of the apparatus while reducing noise due to the apparatus.

Further, the respiratory support apparatus preferably further includes an analyzer that performs frequency analysis on the respiratory waveform of the patient, where the respiratory waveform is detected from the measurement result of the differential pressure sensor, to obtain a signal intensity for each frequency as respiration information.

Thereby, it is possible to appropriately perform control of the operation (that is, the rotation speed) of the blower in accordance with the respiratory flow of a patient.

Further, the respiratory support apparatus is preferably applied to a CPAP apparatus.

Thereby, it is possible to realize the respiratory support apparatus in a more preferable manner.

According to the respiratory support apparatus of the present invention, it is possible to capture the respiratory waveform of a patient more accurately.

### REFERENCE SIGNS LIST

1 Patient
10 Mask
20 Tube
100 CPAP apparatus (respiratory support apparatus)
100A Main body housing
110 Accommodation case
112 Tube connector
113 Intake port
114 Power connector
120 Circuit board
122 Controller
123 Heating controller
124 Respiratory waveform analyzer
125 Communicator
126 Storage
130 Flow path case
131 Blower
132 Flow path
132a Guidance path
132aa to 132ad Bend
132b Blower disposition chamber
150 Base
150A Humidifier
151 Water tank
152 Lid
152a Air inlet port
152b Air exhaust port
153 Heater
154 AC/AD converter
155 Sheet metal member
156 Fan
161 Filter
162 Temperature/humidity sensor
164 Differential pressure sensor
165 Pressure sensor
166 Temperature sensor
168 Temperature sensor
169 Heater
200 External system

1. A respiratory support apparatus, comprising:
a main body housing that includes an intake port and an exhaust port and forms a flow path of air from the intake port to the exhaust port;
a blower that is disposed in the flow path and generates airflow to be delivered into an airway of a patient; and
a differential pressure sensor that is disposed to detect a state of the airflow, includes a first measurement port and a second measurement port, and measures a differential pressure between the first measurement port and the second measurement port, the first measurement port and the second measurement port being disposed in the flow path, wherein:
the flow path includes:
a guidance path that guides the air from the intake port to a position of the blower; and
a blower disposition chamber that is formed such that a flow-path cross-sectional area is expanded from a downstream end of the guidance path,
the first measurement port is disposed in the guidance path, and
the second measurement port is disposed in the blower disposition chamber.

2. The respiratory support apparatus according to claim 1, wherein
the first measurement port is disposed at a position in the guidance path, the position being a predetermined distance away from a connection position between the guidance path and the blower disposition chamber toward a side of the intake port.

3. The respiratory support apparatus according to claim 1, wherein
the second measurement port is disposed at a position in the blower disposition chamber, the position being a position on a side opposite to a connection position between the guidance path and the blower disposition chamber.

4. The respiratory support apparatus according to claim 1, further comprising a controller that controls a rotation speed of the blower based on a respiratory waveform of the patient, the respiratory waveform being detected from a measurement result of the differential pressure sensor.

5. The respiratory support apparatus according to claim 1, wherein
the guidance path guides the air from the intake port to the position of the blower such that the air largely detours in the main body housing.

6. The respiratory support apparatus according to claim 5, wherein:
the blower includes a suction port that opens in a direction intersecting with a guidance direction of the air in the guidance path, and
the blower disposition chamber is formed so as to bend the airflow in the direction intersecting with the guidance direction, the airflow flowing through the guidance path.

7. The respiratory support apparatus according to claim 1, further comprising an analyzer that performs frequency analysis on a respiratory waveform of the patient to obtain a signal intensity for each frequency as respiration information, the respiratory waveform being detected from a measurement result of the differential pressure sensor.

8. The respiratory support apparatus according to claim 1, which is applied to a continuous positive airway pressure (CPAP) apparatus.
